(19) United States
(12) Patent Application Publication
(10) Pub. No.: US 2014/0227731 A1
(43) Pub. Date: Aug. 14, 2014

(54) DRUG FOR ANALYSIS OF WATER TRANSPORT FUNCTION OF MEMBRANE PROTEIN IN BIOLOGICAL TISSUE

(71) Applicant: TAIYO NIPPON SANSO CORPORATION, Tokyo (JP)

(72) Inventors: AKIHIKO MAEDA, Zushi-shi (JP); Hiroshi Taira, Tsukuba-shi (JP); Takayuki Fujimoto, Tokyo (JP); Kiyoshi Nagasawa, Tokyo (JP); Tsuyoshi Matsuda, Tokyo (JP); Yuji Hashiguchi, Tokyo (JP); Akira Nakatani, Tokyo (JP)

(73) Assignee: Taiyo Nippon Sanso Corporation, Tokyo (JP)

(21) Appl. No.: 13/763,883

(22) Filed: Feb. 11, 2013

(51) Int. Cl. G01N 33/50

(52) U.S. Cl. CPC G01N 33/5088
USPC 435/29

(57) ABSTRACT

The present invention is to provide a drug for analysis of a water transport function of a membrane protein in a biological tissue, wherein the abundance of one or both of ¹⁷O water molecules or ¹⁸O water molecules in the drug is greater than their abundance in natural water. Furthermore, the present invention is to provide a method of analysis of a water transport function of a membrane protein in a biological tissue, a usage of the drug for analysis of a water transport function, and a method of diagnosis diagnosing pathologies caused by abnormalities of the water transport function.

This application is a divisional of application Ser. No. 12/532,896 filed Sep. 24, 2009 which in turn is the U.S. national phase of International Application No. PCT/JP2008/055897 filed 27 Mar. 2008 which designated the U.S. and claims priority to Japanese Patent Application No. 2007-084353 filed 28 Mar. 2007, the entire contents of each of which are hereby incorporated by reference.

### TECHNICAL FIELD

The present invention relates to a method of analyzing the water transport function of membrane protein that exists in biomembranes of biological tissue. Here, “water transport function” refers to a function of membrane protein that exists in biomembranes when water molecules are transported via membranes.

### BACKGROUND ART

Water accounts for the greater part of biogenic substance, and plays an important role in preserving the normal functioning of biological tissue. Thus, it is anticipated that elucidation of water transport functions in living organisms will lead to elucidation of the causes of various pathologies, establishment of therapeutic methods, development of diagnostic agents and therapeutic agents, and so on.

In this context, a membrane protein was discovered in 1998 which is involved in water transport as a channel for water molecules in cell membranes, and was named “aquaporin” (hereinafter occasionally referred to as “AQP”). From bacteria to plants, numerous types of aquaporins have been ubiquitously detected.

Aquaporins play a variety of physiological roles in living organisms, and it has been suggested that abnormalities in aquaporins may be related to various types of abnormalities in living organisms.

As to the physiological roles of aquaporins, for example, it has been reported with respect to E. coli that colonies are reduced in size with deficiencies of AQP-Z, suggesting a relation to cellular growth. With respect to Arabidopsis, it has been reported that growth of the root quickens with deficiencies of aquaporin expressed in the root, and it is thought that this is due to an attempt to compensate for deficiencies in water channels by enlargement of the area of the root. With respect to plants, as it becomes impossible to inhibit autogamy when there are deficiencies in the aquaporin of the stigma of the pistil, it has been suggested that the pertinent aquaporin is involved in the growth of the pollen tube at the time of pollination.

On the other hand, knockout mice have been engineered for AQP 1 to AQP 5, and impairment of urinary concentration has been observed in knockout mice of AQP 1, 2, 3, and 4. In particular, with deficiencies of AQP 2, polyuria is conspicuous, and the mice die at about two weeks after birth. Moreover, with deficiencies of AQP 5, impaired secretion of the salivary glands has been confirmed. Disorders in mice due to deficiencies in the remaining AQP 6-9 are still unclear, but the possibility exists that very serious conditions may be exhibited. AQP 1 is also expressed in vascular endothelium; the development of transplanted tumors in AQP 1 knockout mice is poor, and the possibility of involvement in developmental disorders of vasa vasorum has been pointed out. In addition, production of cerebrospinal fluid is significantly inhibited, and it has been reported, for example, that inhibition is on the order of 20-25%. AQP 3 exists in skin, it is known that knockout mice with deficiencies therein have abnormally dry skin, and it is thought that AQP 3 is necessary for maintaining the moisturizing properties of skin. Cerebral edema occurs with difficulty in AQP 4 knockout mice, suggesting that AQP 4 is involved in cerebral edema after cerebral ischemia. As it has been reported that steroid hormones which have heretofore been used to alleviate cerebral edema inhibit the expression of AQP 4, it is possible that AQP 4 may also be involved in a portion of the pharmacological effects.

Given these circumstances, it is extremely important to analyze the water transport functions of membrane proteins such as aquaporin, and there is strong demand for the establishment of such methods of analysis.

With respect to analysis of water transport in cell membranes, some in vitro methods of analysis have heretofore been known. For example, electrochemical techniques and techniques which measure osmotic pressure are being implemented (see Non-Patent Document 1).

Non-Patent Document 1: Kato et al., Plant Cell Engineering 18, “New Developments in Membrane Transport Systems—Pump Transporter Channel Research of Plants,” Shujunsha, (2003) pp. 159-166.

### DISCLOSURE OF INVENTION

### Problem that the Invention is to Solve

As mentioned above, clarification of the membrane permeation function of water in living organisms is extremely important in elucidation of the causes of various pathologies, establishment of therapeutic methods, and development of diagnostic agents and therapeutic agents. However, while conventional technologies exist which are usable in vitro, technologies which can be applied in vivo have yet to be disclosed.

The present invention has been made in light of the foregoing circumstances, and its task is to offer a drug which is well-suited to the acquisition and analysis of information concerning the water transport function and abnormalities of this function with respect to biological tissue in vivo, and which can conduct safe and highly accurate analysis of the water transport function of membrane protein that exists in biological tissue.

### Means for Solving the Problem

In order to solve the aforementioned problem, the present invention offers the following aspects.

(1) A drug for analysis of a water transport function of a membrane protein in a biological tissue, wherein the abundance of one or both of ¹⁷O water molecules or ¹⁸O water molecules in the drug is greater than their abundance in natural water.

(2) The drug for analysis of a water transport function according to (1), wherein said drug for analysis contains deuterium.

(3) A method of analysis of a water transport function of a membrane protein in a biological tissue, comprising introducing the drug for analysis of a water transport function according to (1) or (2) into a biological tissue, after which conducting an image analysis to evaluate a dynamic state of water.

(4) The method of analysis of a water transport function of a membrane protein in a biological tissue according to (3), further comprising evaluating a water transfer state.

(5) The method of analysis of a water transport function of a membrane protein in a biological tissue according to (3) or (4), wherein a substance which acts upon the water transport function of membrane protein is introduced into a biological tissue, after which said drug for analysis is introduced into the biological tissue.

(6) The method of analysis of a water transport function of a membrane protein in a biological tissue according to (5), wherein a substance which inhibits the water transport function of a membrane protein is introduced into the biological tissue as said substance which acts upon the water transport function, after which said drug for analysis is introduced into the biological tissue.

(7) The method of analysis of the water transport function of a membrane protein is introduced into the biological tissue according to (5), wherein a substance which accelerates the water transport function of a membrane protein is introduced into the biological tissue as said substance which acts upon the water transport function, after which said drug for analysis is introduced into the biological tissue.

(8) A usage of the drug for analysis of a water transport function according to (1) or (2), wherein said drug for analysis is used to analysis a water transport function of a membrane protein in biological tissue.

(9) The usage of the drug for analysis of a water transport function according to (1) or (2), wherein said drug for analysis is used to identify a substance that acts upon a water transport function of membrane protein.

(10) A method of diagnosis, comprising introducing the drug for analysis of a water transport function according to (1) or (2) into a biological tissue, after which conducting an image analysis to evaluate a dynamic state of water, and detecting abnormalities in the water transport function of a membrane protein in the biological tissue according to the evaluation of the dynamic state of water.

(11) The method of diagnosis according to (10), wherein an abnormality in the water transport function of a membrane protein in a human biological tissue is detected.

(12) The method of diagnosis according to (10) or (11), wherein a cerebrospinal fluid circulatory disorder is diagnosed.

### Effects of the Invention

In the following description, “water molecules containing one or both of ¹⁷O or ¹⁸O”, are termed “these water molecules,” and water containing “these water molecules” is termed “water with such water molecule content.”

According to the aspect recorded in (1) above, with respect to living biological tissue, it is possible to conduct safe and highly accurate analysis of the water transport function of membrane protein that exists in biological tissue, and to acquire information concerning the water transport function and abnormalities thereof. Moreover, according to the aspect recorded in (2) above, it is possible to acquire more detailed information concerning the water transport function and abnormalities thereof.

According to the aspect recorded in (3) or (4) above, as the dynamic state of water in biological tissue are evaluated by imaging analysis after introducing the drug for analysis recorded in (1) or (2) above into biological tissue, it is possible to conduct detailed identification of the presence or absence of abnormalities and the degree of abnormalities in membrane protein, as well as the existence region of membrane protein, and further to infer the type of the membrane protein.

Moreover, according to the aspects recorded in (5) to (7) above, as the effects of the substances that act upon the water transport function differ according to the individual membrane protein, when the method of (3) above is conducted after differentiating reductions in the water transport function of the individual membrane proteins, it is possible to conduct a more detailed analysis of the water transport function of membrane proteins. Moreover, using known substances (inhibitors or accelerators) that act upon the water transport function as evaluation indicators, if one compares the results from the introduction of these substances and evaluation of water dynamics (conditions of water transfer), and the evaluation results in cases where target substances are introduced, it is possible to evaluate the degree of action (degree of inhibition or degree of acceleration) of the pertinent substances on the water transport function of membrane protein. In cases where the pertinent target substances are unidentified, it is also possible to identify novel substances which act upon the water transport function if conventional chemical analysis techniques and the like are used.

According to the aspects recorded in (8) or (9) above, it is possible to conduct safe and highly accurate analysis with respect to the water transport function of membrane protein that exists in biological tissue, and to acquire information concerning the water transport function and abnormalities thereof.

According to the aspects recorded in (10) to (12) above, it is further possible to conduct safe and highly accurate diagnosis of various types of disorders which originate in abnormalities of the water transport function of membrane protein.

### BRIEF DESCRIPTION OF THE DRAWINGS

FIG. 1 is a graph which shows mean values of signal intensity computed from MRI measurement results in Embodiment 1.

FIG. 2 is a graph which conducts hourly plotting of signal change rates computed from MRI measurement results in Embodiment 2.

### BEST MODE FOR CARRYING OUT THE INVENTION

The present invention is described in detail below.

As water molecules containing oxygen stable isotopes, when water molecules containing deuterium are also included, there exist the 9 types of H₂¹⁶O, HD¹⁶O, D₂¹⁶O, H₂¹⁷O, HD¹⁷O, D₂¹⁷O, H₂¹⁸O, HD¹⁸O, and D₂¹⁸O. In the present invention, one uses one or more types of water molecules selected from a group composed of H₂¹⁷O, HD¹⁷O, D₂¹⁷O, H₂¹⁸O, HD¹⁸O, and D₂¹⁸O. Among these, use of H₂¹⁷O is preferable, because it exhibits particularly excellent effects, and because it is industrially obtainable at low cost.

In the present invention, after the introduction into biological tissue of water with such water molecule content, measurement of the water molecules in the biological tissue or the water molecules that have transited the biological tissue is conducted. Or one may also measure both the water molecules in the biological tissue and the water molecules that have transited the biological tissue. With respect to the transport of water via membrane protein, there are cases where it is conducted between biological tissues, and cases where it is conducted between biological tissue and an external environment, and the water molecules which are to be the subject of measurement may be selected according to purpose. The water transport function of the membrane protein in the biological tissue is then analyzed by conducting measurements of the water molecules. It is also acceptable if isotopic water molecules other than these water molecules are contained in water with such water molecule content, and it is also possible for water molecules other than these water molecules to be included in the water molecules that are measured.

As the content of these water molecules in water that is introduced into biological tissue becomes higher, analysis of greater accuracy is possible. For example, one method is to have the content of one or both of ¹⁷O and ¹⁸O be 0.3 atom % or higher.

In the case of use of ¹⁷O, it has been confirmed from the results of phantom experiments that amplification effects of adequate contrast are obtained with water which has a ¹⁷O content that is higher than the natural abundance ratio by 0.05 atom % or more (Bilgin Keserci et al., Feasibility Study of Oxygen-17 Water as a Cerebrospinal Disease Diagnosis Agent Phantom Study (2006) JSMRM 2006, I84-34PM).

Accordingly, the abundance (content) of either one or both of ¹⁷O and ¹⁸O in the drug for water transport function analysis of the present invention is greater than the pertinent abundance in natural water, and, specifically, can be set to 0.05 atom % or more, and preferably 0.3 atom % or more. As to the upper limit of the abundance of these water molecules, there are no particular limits given that analysis of greater accuracy is possible as the content thereof increases, but this content in the drug for water transport function analysis of the present invention can be prepared within a range of 0.05-90 atom %, and preferably 0.3-90 atom %. Moreover, in order to economically and efficiently manufacture the pertinent drug for water transport function analysis without impairing the effects of the present invention, it is preferable that the pertinent content be 0.05-60 atom %, and 0.3-50 atom % is more preferable.

Other ingredients which do not impair the effects of the present invention and which have biotolerability may be included in the water that is introduced into biological tissue. As the aforementioned other ingredients, there are, for example, buffering agents of buffer solutions and the like of phosphate, acetate, carbonate, citrate, etc.; antioxidant agents such as sulfite, ascorbic acid, and α-tocopherol; thickening agents such as hyaluronic acid and pectin; preservatives such as para-hydroxybenzoic acid esters, chlorobutanol, benzyl alcohol, phenethyl alcohol, dehydroacetic acid, and sorbic acid; tonicity agents such as glucose, D-sorbitol, sodium chloride, potassium chloride, glycerine, D-mannitol, and boric acid; soothing agents such as benzyl alcohol, and so on. These are to be appropriately selected according to the purpose of analysis.

In the present invention, measurement is conducted with respect to these water molecules in biological tissue into which the aforementioned water is introduced and with respect to these water molecules that have transited the aforementioned biological tissue. As for measurement methods, one may cite the nuclear magnetic resonance spectroscopy method (hereinafter “NMR method”), nuclear magnetic resonance imaging method (hereinafter “MRI method”), and mass spectrometry method (hereinafter “MS method”), and these measurement methods may be used alone, or in combinations of two or more.

As water with such water molecule content behaves in the same way as normal water (H₂¹⁶O) relative to biological tissue, it is possible to accurately analyze the water transport function of membrane protein in living organisms.

Among these, the NMR method or MRI method are particularly appropriate, because they have the advantages of possessing a high degree of versatility and accuracy, and facilitating image display of analytical results with respect to measurement of water molecules in biological tissue, and particularly with respect to measurement of water molecules containing ¹⁷O.

In the case of the NMR method or MRI method, when conducting detection which uses ¹H as the detection nucleus, it is difficult to separately measure the spectra of ¹H bonded to ¹⁶O, ¹H bonded to ¹⁷O, and ¹H bonded to ¹⁸O. However, as it is known that ¹H bonded to ¹⁷O has a shorter transverse relaxation time (T2) of hydrogen atoms than ¹H bonded to ¹⁶O, it is sufficient if this time difference is detected. Moreover, with the NMR method or MRI method, one may conduct direct detection using the ¹⁷O in water molecules containing ¹⁷O as the detection nucleus.

With the NMR method and MRI method, detection of ¹H is preferable from the standpoints of ease of operation and high sensitivity, but if water is used in which water molecules containing ¹⁷O are concentrated, these water molecules can be detected to a high degree of accuracy even by conducting direct detection of ¹⁷O.

As ¹⁷O changes the bonding conditions of neighboring atoms due to its nuclear spin, such atoms cause changes in relaxation time during nuclear magnetic resonance. Accordingly, when measuring water molecules containing ¹⁷O by the NMR method or MRI method, it is further preferable to detect changes in relaxation time of the atoms within the biological tissue. Here, “atoms within biological tissue” refers to atoms that configure biological tissue or atoms that are contained in biological tissue; either is acceptable so long as relaxation time is changed due to the effects of ¹⁷O.

On the other hand, the MS method is preferable for measurement of water molecules that have transited biological tissue, particularly for water molecules containing ¹⁸O. If the MS method is used, it is possible to acquire information concerning the abundance ratio of oxygen stable isotopes of water molecules based on differences in the mass of these molecules, and quantitatively confirm the existence of these water molecules.

Thus, it is possible to measure these water molecules in the foregoing manner. By confirming the existence region of these water molecules in biological tissue, it is also possible to detect the dynamics of these water molecules in biological tissue.

Now, “dynamic state of water” in the present invention is a concept which includes the modality of water transfer due to active transport of water by membrane protein in the region of interest of biological tissue, and aspects that are measurable by the drug for analysis and methods of analysis of the present invention. At the same time, the “water transfer conditions” of the present invention signify water transfer conditions between the membrane proteins of a certain region of interest and another region of interest, and among multiple membrane proteins in multiple regions of interest, and the modalities of water transfer by passive transport such as osmosis and diffusion of water.

For example, in the context of effects which are exercised on the water transport function of membrane protein due to external stress that is imposed on the living organism or due to substances that act upon the water transport of membrane protein such as inhibitors and accelerators, it is possible to analyze the water transport function in relation to membrane protein if one is able compare the dynamic state of water in biological tissue.

Particularly with respect to the NMR method or MRI method, the existence of water molecules containing ¹⁷O can be confirmed with greater clarity by obtaining images which highlight the transverse relaxation time (T2) of hydrogen atoms. Moreover, the amount of these water molecules can be quantified if a calibration curve is prepared.

In the present invention, there are no particular limitations on the membrane proteins which are the subject of analysis so long as they are involved in water transport via these membranes. For example, one may cite membrane proteins which have channel functions related to the transport of water into and out of biomembranes. In particular, the below-mentioned aquaporins may be enumerated.

The present invention is well-suited to the obtainment of information concerning the water transport function of these membrane proteins, their abnormalities, or substances or environmental stresses that act upon water transport.

So long as it is of biological origin, the biological tissue which is the subject of analysis may be microbe-derived, animal-derived, plant-derived, etc.

As animals, one may cite, for example, vertebrates, and invertebrates such as prokaryotes, eukaryotes, mollusks, annelida, and arthropods. As plants, one may cite, for example, gymnosperm and angiosperm.

There are no particular limitations on the biological tissue, but tissue suggesting the existence of membrane protein having a water transport function is preferable. For example, one may cite cells, callus, embryonic tissue, xylem tissue, cuticle, meristematic tissue, epidermal tissue, conductive tissue, mechanical tissue, connective tissue, muscular tissue, neural tissue, and tissue or the like containing body fluid, blood, lymphatic fluid, tissue fluid, coelomic fluid, cerebrospinal fluid, synovial fluid, or ocular water.

More specifically, one may cite leafage, stalk, duct, root hair, vacuole, stoma, germ layer, branchia, spiracle, brain, heart, lung, liver, pancreas, gall bladder, kidney, urinary bladder, intestine, eye, nasal cavity, salivary gland, trachea, spinal marrow, testis, skin, muscle, erythrocyte, leukocyte, and so on.

Providing such biological tissue for analysis, it is possible by means of the present invention to acquire information concerning the water transport function of membrane protein, abnormalities thereof, and diseases related to such abnormalities. Particularly with respect to the aquaporins as membrane protein, there are known to be many which have similar functions and structures such as aquaporin 0-12. These exist widely in nature, and have been shown to be involved in various water transport function abnormalities of biological tissue. There is also a strong possibility that aquaporins are contained in each of the biological tissues that are specifically cited above.

In the case of animals, for example, it is known that cerebrospinal fluid is produced in the region called the choroid plexus of the cerebral ventricle, that it runs through the spinal cord, and that it is finally absorbed by the superior sagittal sinus in the falx cerebri. Aquaporin 1 exists in the choroid plexus, and aquaporin 4 in the superior sagittal sinus. When abnormalities such as deficiencies or the like arise with respect to these aquaporins, disorders occur with respect to production and absorption of cerebrospinal fluid, and it has been suggested that this may lead to abnormalities in the circulation of body fluids.

On the other hand, it is not possible to conduct intracerebral testing using drugs, because the blood-brain barrier protects the brain from invasion by chemical substances, and direct observation is also difficult, because the tissue is microscopic. Accordingly, functional analysis of aquaporin within the brain has not been adequately conducted. In contrast, as these water molecules that are used in the present invention behave in the same way as normal water (H₂¹⁶O) relative to biological tissue, it is possible to safely and easily conduct analysis of the water transport function in the brain. After introduction into the brain, highly accurate measurement is possible from outside of the body. Accordingly, it is possible to quantitatively measure the circulation of body fluid, e.g., the amount of production and amount of absorption of cerebrospinal fluid in the brain.

In the case of plants, for example, it is known that conductive tissue such as ducts is involved in the water transport. As it is highly likely that aquaporin exists in the vicinity of these ducts, regions which have ducts are well-suited to undergo analysis.

There are no particular limitations on methods for introducing water with such water molecule content into biological tissue, and selection may be appropriately made according to the type and condition of the biological tissue that is to undergo analysis.

For example, to conduct introduction into the biological tissue of living plants, the aforementioned water may be absorbed from root hair, or it may be absorbed during culture. Or the biological tissue may be immersed in the aforementioned water. In the case of immersion in water, one may cite, for example, the method of immersing segment, callus, epidermal tissue, epithelial tissue, cortical layer, endothelium, etc. Furthermore, it is also acceptable to directly inject the aforementioned water into the biological tissue. In the case of injection of water, injection may be conducted, for example, into conductive tissue, mesophyllic tissue, central cylinder, seed, germinal tissue, etc. Direct injection is optimal in cases where the biological tissue has no water absorption capability or a weak water absorption capability.

On the other hand, to conduct introduction into biological tissue of animals, one may cite, for example, the method of introduction by drinking, feeding, or inhalation, and the method of absorption through skin. As in the case of plants, one may directly inject the water into the biological tissue, or one may immerse the biological tissue in the water. Furthermore, it is also acceptable to introduce the aforementioned water into biological tissue which is the subject of analysis by conveying it by body fluid by injection, for example, into the peritoneal cavity, the digestive organs, blood, or the spinal cord. When conducting intravascular injection, either veins or arteries are acceptable.

The administration amount of the aforementioned water is to be appropriately selected according to the content of these water molecules, and the type, condition and the like of the biological tissue. For example, when conducting introduction into biological tissue of a living animal, it is preferable to conduct administration so that physiological conditions within the body of the animal are not significantly changed so as to enable analysis to be more accurately conducted. Specifically, it is preferable to administer water in which the content of these water molecules is 0.05-90 mass % at 0.01-10 ml per 1 kg of body weight.

Apart from the biological tissue of living animals, with respect to, for example, the biological tissue of plants, there are no particular limitations on the administration amount of the aforementioned water, and it may be adjusted so as to enable accurate measurement.

In the present invention, before introducing water with such water molecule content into biological tissue, it is preferable to introduce substances which act upon the water transport function into the pertinent biological tissue, such as substances which inhibit or substances which accelerate the water transport function of the membrane protein which is the subject of analysis. By making concomitant use of substances that act upon the water transport function in this way, it is possible to conduct a more detailed analysis of the water transport function of membrane protein by making comparisons with analytic results obtained in the case where, for example, substances that act upon the water transport function are not used.

To cite a specific example, it may be inferred that membrane protein is normal if water molecules are transported when inhibitors are not present, and if, inversely, water molecules are not transported when inhibitors are present. For example, by comparing the degree of acceleration in the water transport function between individual solids with respect to specific accelerators of the water transport function, it is possible to evaluate individual variability in sensitivity relative to accelerators, and also to clarify the relation with phenotypes (e.g., changes in the symptoms of each of the below-mentioned diseases) of each individual. By identifying the region(s) where such phenomena are observed, it is possible to conduct detailed identification of the existence regions of membrane protein, and analyze the in vivo dynamics of substances that act upon the water transport function. In addition, with respect to substances which act upon the water transport function such as inhibitors and accelerators, it would be possible to infer the type of membrane protein if there are substances which have particularities with respect to membrane proteins.

Furthermore, using known substances that act upon the water transport function as evaluation indicators, it is possible by the method of analysis of the present invention to evaluate the degree of action (degree of inhibition or acceleration) of a target substance relative to the water transport function of membrane protein from the results of evaluation of water dynamics and the conditions of water transfer obtained by the introduction of that substance, and from comparison with evaluation results obtained in the case of introduction of other target substances. For example, if a drug which affects the water transport function and which has already been clinically employed is used as an evaluation indicator, it would be possible to select substances as candidates for pharmaceuticals having a higher degree of effectiveness relative to the water transport function, enabling development of pharmaceuticals that have superior clinical effects. Otherwise, if such target substances are not identified, it would also be possible to identify novel substances that act upon the water transport function using known chemical assay techniques and the like.

Here, the “degree of effectiveness relative to the water transport function” which is possessed by substances that act upon the water transport function of membrane protein is measured by the evaluation of “water dynamics” and “conditions of water transfer” in the present invention.

As substances that act upon the water transport function of membrane protein, one may cite inhibitors, accelerators, and the like. Specifically, such substances that act upon the water transport function of membrane protein may be substances that act directly on aquaporin to inhibit or accelerate the water transport function, or they may be substances that indirectly inhibit or accelerate the water transport function of membrane protein such as aquaporin by acting upon other biomolecules involved in the water transport function (including, e.g., substances that act upon the water transport function by promoting or inhibiting the expression of aquaporin in biological tissue). In the case where the substance that acts upon the water transport function of membrane protein is an inhibitor, it may, for example, be a substance which inhibits the transport of water molecules by directly acting upon aquaporin, or it may be a substance which antagonistically inhibits water molecules. Furthermore, as substances that accelerate the water transport function of membrane protein, one may cite substances (channel openers) and the like that have an opening effect on aquaporin.

There are no particular limitations on the types of substances that act upon the water transport function in the present invention, so long as they do not hinder the water transport function analysis of the present invention. For example, artificially synthesized compounds or peptides or the like are acceptable, as are biologically derived natural substances. More specifically, silver ions (silver nitrate, silver sulfadiazine, etc.), mercury ions (mercury chloride, p-chloromercurybenzene sulfonate, etc.), gold ions (tetrachloroauric acid, etc.) and so on are known as substances that directly inhibit aquaporin. On the other hand, as substances that accelerate the water transport function, substances that have an opening effect on aquaporin 5 (Japanese Unexamined Patent Application Publication, First Publication No. 2001-114698) have been suggested.

In this case, it is known, for example, that mercury compounds such as mercury chloride do not act upon aquaporin 4, and that the relation of the active substance in biological tissue to the speed of the water transport that it affects varies according to the type of active substance that is used. If the specificity of such activity is utilized, it is also be possible to infer type with respect to membrane protein which, as mentioned above, is specifically expressed in the organs and biological tissue of the region of interest and the specific types of cells and the like that compose the pertinent tissue.

Furthermore, by comparing the results obtained from analysis according to the present invention using conventional substances whose properties are well-known like those mentioned above with evaluation results obtained using other target substances which are the subject of analysis, it is possible to grasp the degree of effectiveness (degree of inhibition or acceleration) of the pertinent target substances relative to the water transport function. More specifically, if substances are selected according to the drug for analysis and method of analysis of the present invention which have greater effectiveness in comparison to existing inhibitors or accelerators, it would be possible to develop pharmaceuticals which can be expected to have better therapeutic effects than existing pharmaceuticals, more effective agrochemicals, and so on.

The administration amount of a substance such as an inhibitor that acts upon the water transport function may be suitably selected according to the type of substance and the type of the membrane protein that is the subject of analysis. In the case where the membrane protein is an aquaporin, one may use, for example, silver ions, mercury ions, and gold ions as inhibitors of the water transport function. It is then preferable to conduct administration to the biological tissue as an aqueous solution with a concentration of silver ions of 100 μmol/L or higher, or mercury ions and gold ions of 1 mmol/L or higher.

In the present invention, after introduction of water with such water molecule content into biological tissue, the water transport function of membrane protein can be analyzed by conducting multiple measurements of water molecules, that is, by conducting so-called temporal measurement, and conducting an analytical process over time.

In particular, to determine the presence or absence of abnormalities in membrane protein, it is important to conduct the measurement of quantity and movement speed of water molecules on an organ level or cellular level even within biological tissue.

In the aforementioned types of membrane protein, there are water molecules containing deuterium (hereinafter referred to as “D”), that is, molecules that do not transmit deuterium water or that have a low efficiency of transmission of deuterium water. When the nuclear magnetic resonance method, nuclear magnetic resonance imaging method, or mass spectrometry method is used with D as the detection nucleus, it is possible to conduct comparative observation of deuterium water and these water molecules within biological tissue. That is, when analyzing the water transport function of membrane protein, it is possible to simultaneously analyze the dynamic state of water containing deuterium water and these water molecules by having these water molecules contain an appropriate amount of deuterium water. In this case, one can use the difference in the respective transmittance rates in the membrane protein. In the case where this more detailed analysis becomes necessary, it is preferable to use a water transport function analysis drug containing these water molecules and D.

Deuterium water refers to D₂¹⁶O, HD¹⁶O, D₂¹⁷O, HD¹⁷O, D₂¹⁸O, or HD¹⁸O, and any of these may be used.

In the present invention, as the water molecules which transit the biological tissue that is provided for analysis, one may cite water molecules which exist outside the aforementioned biological tissue as a result of extraction from transpiration, evaporation, perspiration, excretion, urination, secretion, respiration, osmosis, or resection; extraction from epidermal tissue; extraction from epithelial tissue; extraction from conductive tissue; extraction from peritoneal cavity; extraction from digestive organ; extraction from muscle; extraction from blood vessels; or extraction from spinal cord.

By measuring water molecules that have transited biological tissue, one can acquire not only information about the water transport function of membrane protein, but also about the circulation of water, which is useful for understanding the roles of membrane protein in a living organism

As regards abnormalities in living organisms that originate in abnormalities of the water transport function of membrane protein, one may cite with respect to plants, for example, wilting, water absorption abnormalities, transpiration abnormalities, cellular growth abnormalities, and so on.

With respect to animals, one may cite, for example, cellular growth abnormalities, urinary concentration abnormalities, endocrine secretion abnormalities, moisture retention abnormalities, blood pressure abnormalities, body fluid circulation abnormalities, digestive system abnormalities, circulatory system abnormalities, respiratory system abnormalities, urinary system abnormalities, reproductive system abnormalities, endocrine secretion system abnormalities, sensory system abnormalities, central nervous system abnormalities, motor system abnormalities, reduction in salivary secretion, cataracts, nephrogenic diabetes insipidus, multiple-cyst renocutaneous dryness, and so on. Among these, with respect to human beings, one may cite, for example, cerebrospinal fluid circulatory disorders, edematous disorders, dry eye, Sjogren's syndrome, and so on.

The present invention is particularly well-suited for use in the inspection and diagnosis of these various types of disorders.

Abnormalities in the water transport function are triggered by the occurrence of abnormalities in membrane protein in biological tissue. Abnormalities in membrane protein are caused by the incurrence of various types of disease and environmental stress.

Diseases are contracted, for example, due to invasion by bacteria, fungi, virus, or nematode.

As environmental stresses, one may cite, for example, osmotic pressure stress, saline stress, xerantic stress, temperature stress, optical stress, oxygen stress, nutritional stress, noise stress, ultraviolet stress, water stress, nutrient stress, edaphic stress, or electrical stress. Plants are particularly susceptible to environmental stress.

Accordingly, it is also possible to evaluate the level of disease and environmental stress incurrence by evaluating the water transport function of membrane protein.

According to the present invention, it is possible to evaluate the effects exercised on the water transport function with respect to known substances or environmental stresses that act upon the water transport function of membrane protein as described above, and it is also possible to evaluate and identify whether or not completely unknown substances or environmental stresses act upon the water transport function of membrane protein.

In the case where it is evaluated whether or not a substance of one's choice acts upon the water transport function of membrane protein, after introduction of, for example, one or more types of target substance as the subject substance into biological tissue, the drug for water transport function analysis of the present invention is also introduced into the biological tissue, enabling measurement of these water molecules as described above. By comparing the water dynamics from the imaging analysis obtained by this measurement with results obtained by measurement when the pertinent subject substance has not been added, it is possible to determine whether or not the subject substance acts upon the water transport function of membrane protein. Moreover, as mentioned above, it is also possible to evaluate the degree of activity relative to the water transport function of membrane protein using known compounds or the like as evaluation indicators. In the case where a substance or the like which has been evaluated as acting upon the water transport function of membrane protein is an unknown substance, it is also possible to identify the novel substance and determine its structure using known chemical analysis techniques, molecular biological techniques, and so on.

Otherwise, the types of these subject substances and the types of the environmental stresses that are subjected to testing are the same as the aforementioned substances and environmental stresses that act upon the water transport function of membrane protein.

According to the aforementioned analysis of the water transport function of membrane protein as described above, it is possible to conduct screening of novel substances such as inhibitors and accelerators that act upon the water transport function of membrane protein, and conduct application as a screening technology for substances to serve as candidates for pharmaceuticals, agrochemicals, or the like. In addition, for example, it is also possible to conduct application in identification of environmental stresses that are related to specific diseases. Accordingly, the present invention is also useful in the fields of development of novel pharmaceuticals and agrochemicals, and preventive medicine pertaining to lifestyle-related diseases and the like.

### WORKING EXAMPLES

Below, the present invention is described in further detail by means of specific working examples. However, the present invention is not limited in any way by the working examples shown below.

### Working Example 1

### Detection of Aquaporin in Radish

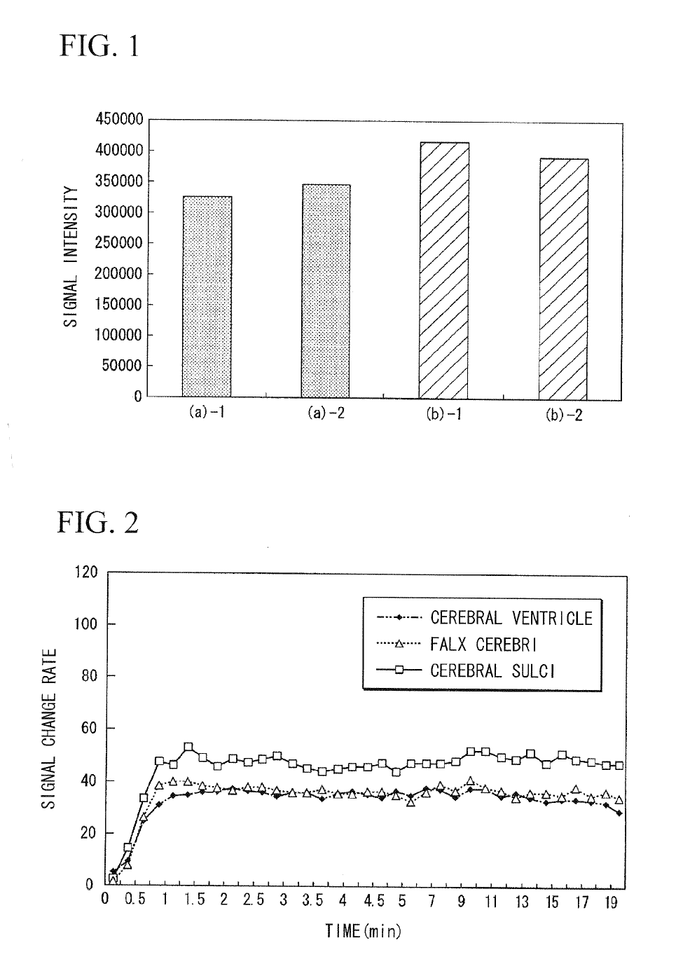

Using (a) water containing H₂¹⁷O so that ¹⁷O concentration was 5 atom %, and (b) injection solvent, stick-shaped radishes with a diameter of approximately 14.5 mm were immersed in these for 20 hours. Next, after extraction from these, the radish was sliced for analytical samples of MRI, and MRI measurement was conducted using ¹H as the detection nucleus. In MRI measurement, the magnetic field intensity was 1.5 T, TE was 177.7 ms, and TR was 5318.7 ms; with respect to the imaging method, T2-weighted images were obtained by the Fast Spin Echo (FSE) method, the number of echoes during imaging was 8, the number of integrations was 4, FOV was 64 mm×64 mm, and the matrix was 128×256. At that time, samples of injection solvent only were subjected to measurement and imaging in the same way for purposes of comparison and contrast. In addition, at that time, regions of interest were established in two places (considered as (a)-1 and (a)-2 when (a) was used, and as (b)-1 and (b)-2 when (b) was used) so as to sandwich the duct of the radish, and signal intensity was compared in these regions of interest. The graph for these is shown in FIG. 1.

With respect to the results of imaging, as shown in FIG. 1, the signal intensities of the radish slice immersed in (a) ((a)-1: 325554, (a)-2: 346196) were smaller than the signal intensities of the radish slice immersed in (b) ((b)-1: 416177, (b)-2: 392158), confirming the difference in signal intensities observed with T2 relaxation accompanying H₂¹⁷O detection. This showed that water transport due to aquaporin that exists in the vicinity of connective tissue was detected, thereby confirming that analysis of the water transport function of aquaporin is possible in accordance with the present invention.

### Reference Example 1

Using radish that was cut into round slices with thicknesses of 6-10 cm, the bottom faces of the round slices were immersed in red food coloring water for 1-16 hours, As the red food coloring, Red No. 102 (chemical name: 7-hydroxy-8-(4-sulfonaphthylazo)-1,3-naphthylenedisulfonic acid=trisodium salt=1½ hydrate) was used. When one hour had elapsed, the red food coloring had soaked into the connective tissue at the periphery of the round slices from the bottom face.

However, even with round radish slices that were immersed in red food coloring water for 16 hours, sites permeated by red food coloring water were not found anywhere other than the connective tissue. It would seem that this was because aquaporin does not allow transmission of high molecular compounds such as red food coloring, and therefore permeation of red food coloring from connective tissue to other areas did not occur.

From the foregoing, it is suggested that aquaporin is involved in much of the water transport apart from connective tissue, and in particular that most of the water transport between connective tissue and peripheral areas is conducted through aquaporin.

### Working Example 2

### Detection of Aquaporin in Canine Brain

With respect to a male beagle dog with a body weight of 5 kg, nuclear magnetic resonance imaging was conducted on brain using ¹H as the detection nucleus. Using a standard quadrature head coil, imaging was conducted by a 3T MRI scanner (SignaExcite, GE Healthcare) as the apparatus. With respect to the imaging method, the FSE method was used (TR/TE=3000/120 ms, ETL=64, number of slices=4, slice thickness=5 mm, BW=31.25 kHZ, FOV=120×120 mm, matrix=256×128, scan time=15 sec). 10 ml of water containing H₂¹⁷O so that ¹⁷O concentration was 40 atom % were administered for 40 seconds at fixed speed into the femoral vein, and imaging was conducted for 15 second intervals from 45 seconds before administration until 5 minutes after administration, and subsequently at 1 minute intervals until 19 minutes after administration. The dog was under anesthesia, and the partial pressure of carbon dioxide was controlled to 40 mm HG at 2-4% CO₂. Regions of interest were established in the cerebral ventricle in which the choroid plexus that produces cerebrospinal fluid exists, in the falx cerebri in which the superior sagittal sinus that absorbs cerebrospinal fluid exists, and in the cerebral sulci. Subtraction of ¹H signal intensity before administration of H₂¹⁷O from ¹H signal intensity after administration was conducted, and the post-administration signal change rate was computed. The signal change rate at each time was plotted, and a graph was created. The results are shown in FIG. 2.

In an early period from several seconds after administration until several tens of seconds later, signal changes that would seem to be due to the inflow of H₂¹⁷O into the cerebral ventricle were confirmed. With the passage of time, similar signal changes were also observed in the cerebral sulci and the falx cerebri. Such rapid transport of water molecules within a living organism is unknown other than via membrane protein, and the inflow of H₂¹⁷O into the cerebral ventricle reflects the transport of water by aquaporin. These results shows that H₂¹⁷O flows together with cerebrospinal fluid into the cerebral ventricle by means of aquaporin 1, subsequently passes through the cerebral sulci after transiting the spinal cord, and finally reaches the falx cerebri.

From the foregoing, it was confirmed by the present invention that analysis of the water transport function of aquaporin is possible.

In the present invention, as measurement of water molecules without use of radioactive isotopes is possible by the NMR method, MRI method, MS method or the like, and as there is no risk of exposure to bombardment during measurement, safety is excellent. Measurement-dedicated equipment is also unnecessary, and as measurement can be simply conducted using general-purpose equipment, the cost burden is small.

Moreover, as water with such water molecule content has almost the same physical properties as ordinary water (H₂¹⁶O), and has the same behavior in biological tissue, the water transport function of membrane protein can be accurately analyzed in normal living organisms.

### INDUSTRIAL APPLICABILITY

As the water transport function of membrane protein in living organisms plays a universally important role in biology, the present invention which enables such analysis has the possibility of being utilized in all fields related to living organisms. For example, in the pharmaceutical field, it could be utilized to: elucidate the causes of various diseases such as urinary disorders, digestive juice secretory disorders, cerebral disorders involving cerebrospinal fluid, and cerebral edema; establish therapeutic methods; develop diagnostic agents and therapeutic agents; develop medical imaging analysis, diagnostic imaging, and the equipment related thereto, and so on. In the agricultural field, it could be used to develop crops with high water-use efficiency, agrochemicals, and so on.

1.-2. (canceled)

3. A method of analysis of a water transport function of a membrane protein in a biological tissue, comprising
introducing drug for analysis of a water transport function of a membrane protein into a biological tissue, and thereafter
conducting an image analysis to evaluate a dynamic state of water to determine if the abundance of one or both of ¹⁷O water molecules or ¹⁸O water molecules in the drug is greater than their abundance in natural water.

4. The method of analysis of a water transport function of a membrane protein in a biological tissue according to claim 3, further comprising
evaluating a water transfer state.

5. The method of analysis of a water transport function of a membrane protein in a biological tissue according to claim 3, wherein
a substance which acts upon the water transport function of membrane protein is introduced into a biological tissue, after which said drug for analysis is introduced into the biological tissue.

6. The method of analysis of a water transport function of a membrane protein in a biological tissue according to claim 5, wherein
a substance which inhibits the water transport function of a membrane protein is introduced into the biological tissue as said substance which acts upon the water transport function, after which said drug for analysis is introduced into the biological tissue.

7. The method of analysis of the water transport function of a membrane protein is introduced into the biological tissue according to claim 5, wherein
a substance which accelerates the water transport function of a membrane protein is introduced into the biological tissue as said substance which acts upon the water transport function, after which said drug for analysis is introduced into the biological tissue.

8.-12. (canceled)
